SEQUENCE LISTING (6 sequences)

SEQ ID NO: 1 - DNA, 18 bp, Artificial Sequence
  Synthetic
acgagtagatgctcaata

SEQ ID NO: 2 - DNA, 19 bp, Artificial Sequence
  Synthetic
ggaataatgacgctatgat

SEQ ID NO: 3 - DNA, 25 bp, Artificial Sequence
  Synthetic
ccaatctaacttccacataccatct

SEQ ID NO: 4 - DNA, 22 bp, Artificial Sequence
  Synthetic
tgatgcgggttgtgttaattga

SEQ ID NO: 5 - DNA, 33 bp, Artificial Sequence
  Synthetic
tccacatctcattaaatttttaaattatacaca

SEQ ID NO: 6 - DNA, 20 bp, Artificial Sequence
  Synthetic
agagcatttaagattatgcg

(19) United States
(12) Patent Application Publication
(10) Pub. No.: US 2013/0052690 A1
(43) Pub. Date: Feb. 28, 2013

(54) PCR PRIMER AND METHOD FOR REDUCING NON-SPECIFIC NUCLEIC ACID AMPLIFICATION USING A PHOTOLABILE PROTECTING GROUP

(71) Applicants: Sung-min CHI, Hwaseong-si (KR); Sung-ouk JUNG, Hwaseong-si (KR); Sun-ok JUNG, Seongnam-si (KR); Joon-ho KIM, Seongnam-si (KR)

(72) Inventors: Sung-min CHI, Hwaseong-si (KR); Sung-ouk JUNG, Hwaseong-si (KR); Sun-ok JUNG, Seongnam-si (KR); Joon-ho KIM, Seongnam-si (KR)

(73) Assignee: SAMSUNG ELECTRONICS CO., LTD., Suwon-si (KR)

(21) Appl. No.: 13/584,524

(22) Filed: Aug. 13, 2012

(30) Foreign Application Priority Data
Aug. 22, 2011 (KR) 10-2011-0083581

(51) Int. Cl. C07H 21/04; C07H 19/10; C12M 1/42; C07H 19/20; C12N 9/12; C12P 19/34

(52) U.S. Cl. 435/91.2; 536/24.33; 435/194; 435/292.1; 536/26.7; 435/286.1; 536/26.8

(57) ABSTRACT

A primer or primer pair comprising a 3′-photolabile group, and a method of amplifying nucleic acids with controlled polymerization using the primer or primer pair, as well as related compositions, kits, and device for amplifying nucleic acids.

### CROSS-REFERENCE TO RELATED APPLICATIONS

This application claims the benefit of Korean Patent Application No. 10-2011-0083581, filed on Aug. 22, 2011, in the Korean Intellectual Property Office, the disclosure of which is incorporated herein in its entirety by reference.

### BACKGROUND

Techniques for amplifying nucleic acids that are contained in a sample in a very small quantity have been efficiently used in biotechnology, for example, molecular diagnosis of a variety of inherited diseases and infectious diseases, mass production and cloning of nucleic acids used in recombinant DNA technology, determination of sequence, and the like. Examples of known techniques for amplifying nucleic acids include polymerase chain reaction (PCR), strand displacement amplification (SDA), rolling circle amplification (RCA), transcription mediated amplification (TMA) based on transcription and reverse transcription, and nucleic acid sequence-based amplification (NABSA). In general, a method of amplifying nucleic acids includes annealing a primer to a template and elongating the primer by using a specific nucleic acid polymerase, which may vary according to the amplification techniques.

In nucleic acid amplification, specificity is determined by annealing stringency of a primer that binds to a targeted nucleic acid sequence, wherein the annealing stringency depends on annealing temperature. In general, as the annealing temperature increases, the possibility of specific annealing to a completely matching template increases, resulting in increasing amplification specificity. As the annealing temperature decreases, mismatching between the template and the primer increases, resulting in an increase in non-specific amplification. Ingredients for gene amplification are mixed at room temperature before an initial denaturation of nucleic acids. Nucleic acids may form various secondary structures at a temperature less than their thermal denaturation temperature. For example, a template nucleic acid strand may form a hairpin loop and a primer may form a dimer. Accordingly, mispriming with low stringency may occur. Since a polymerase is active at room temperature, such mispriming increases non-specific amplification at a temperature less than the thermal denaturation temperature. Products of non-specific amplification reduce accuracy of detection of targeted genes and function as competitive inhibitors, and thus reducing amplification efficiency. In particular, non-specific amplification has been a matter of concern in the detection of low copy number target DNA, amplification of a small amount of a DNA sample, and multiplex PCR using more than one primer pair.

### SUMMARY

Provided is an oligonucleotide primer or primer pair to which a photolabile protecting group is attached.

Also provided is a method of amplifying nucleic acids including controlling polymerization by light irradiation using an oligonucleotide primer to which a photolabile protecting group is attached in order to reduce non-specific amplification.

A composition, dried product, and kit useful for amplifying nucleic acids also is provided, which include an oligonucleotide primer or primer pair to which a photolabile protecting group is attached.

A device for amplifying nucleic acids including a light-transmitting sample receiving unit and a light-irradiating unit also is provided.

In addition, a phosphoramidite nucleoside monomer is provided, which includes a photolabile protecting group.

### BRIEF DESCRIPTION OF THE DRAWINGS

These and/or other aspects will become apparent and more readily appreciated from the following description of the embodiments, taken in conjunction with the accompanying drawings in which:

FIG. 1 is a diagram illustrating a process of elongating a nucleotide chain by removing a protecting group by irradiation of light in a polymerization of nucleic acids using an oligonucleotide primer having a 3′-hydroxy group to which a photolabile protecting group is attached;

FIG. 2 is a diagram illustrating a process of preparing a photolabile oligonucleotide primer using a 5-phosphoramidite nucleoside having a 3′-hydroxy group to which a photolabile protecting group is attached; and

FIG. 3 is a diagram illustrating a process of elongating of a nucleotide chain by removing a protecting group by irradiation of light in a polymerization of nucleic acids using an oligonucleotide primer having a phosphoramidite nucleoside having a 3′-phosphate group to which a photolabile protecting group is attached.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. Expressions such as “at least one of,” when preceding a list of elements, modify the entire list of elements and do not modify the individual elements of the list.

According to an embodiment of the present invention, provided is a method of amplifying nucleic acids, the method including: providing reactants for nucleic acid amplification which include an oligonucleotide primer or primer pair having a 3′-hydroxy group protected by a photolabile protecting group; and exposing the reactants for nucleic acid amplification to electromagnetic radiation in a wavelength range capable of removing the photolabile protecting group in order to reduce non-specific amplification of the nucleic acid.

The oligonucleotide primer includes a priming sequence that complementarily binds to a targeted nucleic acid sequence, wherein a 3′-hydroxy group of the 3′-terminal nucleoside of the priming sequence is protected by a photocleavable photolabile protecting group.

The term “primer” used herein refers to a natural or synthetic oligonucleotide including at least one of ribonucleotide, deoxyribonucleotide, and nucleotide analogues. The primer functions as a starting point for the synthesis of a nucleic acid that is complementary to the primer and elongated from the primer under conditions in which the synthesis of the nucleic acid induced, for example, the presence of nucleotide and nucleic acid polymerase, temperature, and pH.

The nucleic acid monomer that constitutes the photolabile primer may be deoxyribonucleotide, ribonucleotide, nucleotide analogues, or any mixture thereof which do not deteriorate the functions of the primer. The nucleotide analogues are compounds having similar structural features to natural nucleotides or nucleosides so as to be hybridized with natural oligonucleotides when inserted into the oligonucleotides. These analogues may be compounds derived by substituting or modifying a base, a ribose or a portion of a phosphodiester moiety of a natural nucleotide or nucleoside. For example, the analogues may be compounds prepared by protecting at least one nitrogen atom in purine and pyrimidine bases using dimethoxytrityl, benzyl, tert-butyl, isobutyl, or the like, compounds prepared by substituting a 2′-hydroxy group of ribose with a halogen atom or an aliphatic alkyl group, or a nucleotide protected by a functional group such as ether, but are not limited thereto. For example, the monomer that constitutes the primer is a natural deoxynucleoside monophosphate (dNMP) such as dAMP, dGMP, dCMP, and dTMP. In addition, the primer may be single-stranded for increasing efficiency of amplification.

The term “targeted nucleic acid” or “template” refers to a nucleic acid to be amplified. The term “nucleic acid” refers to a single-stranded or double-stranded deoxyribonucleotide or ribonucleotide polymer, or any combination thereof, and also includes a natural nucleotide and a synthetic nucleotide. Thus, according to the method of amplifying nucleic acids, according to the current embodiment, natural nucleic acid molecules, such as, nucleic acids of prokaryotic cells, nucleic acids of eukaryotic cells, viral nucleic acids, or viroid nucleic acids, known nucleic acid analogue molecules, or chemically synthesizable nucleic acid molecules may be amplified.

According to the current embodiment, the targeted or template nucleic acids may have a sequence derived from mRNA. In this case, the amplification may be performed using a cDNA, obtained by reverse transcription of mRNA, as a template. For the reverse transcription, an oligonucleotide dT primer that is hybridized with a poly A tail of the template mRNA is used. The oligonucleotide dT primer consists of dTMP, at least one dTMP may be replaced with another dNMP provided that the dT primer functions as a primer. The reverse transcription may be performed by using a reverse transcriptase having RNase H activity.

According to the current embodiment, the targeted or template nucleic acid may be double-stranded or single-stranded. If the template is a double-stranded nucleic acid, a denaturation may further be performed to separate the double-stranded nucleic acid into a single-stranded or a partially single-stranded nucleic acid. The denaturation may be performed by using heat, alkali, form amide, urea, glyoxal, enzyme (for example, helicase), and a binding protein, but is not limited thereto. For example, the double-strands may be separated by a thermal denaturation at about 80 to about 105° C.

The oligonucleotide primer used herein has a nucleotide sequence complementary to a portion of a targeted nucleic acid molecule, i.e., a priming sequence. The term “complementary” used herein refers to “substantially complementary”, which indicates that a primer is sufficiently complementary to a template or targeted nucleic acid sequence to be selectively hybridized with the template or targeted nucleic acid sequence under an annealing condition. Thus, the primer may have at least one mismatch with the template as long as it functions as a primer. In particular, a 5′-terminal of the primer relatively less influences on specificity of annealing to a targeted sequence. Thus, the 5′-terminal may be modified so as to have an additional sequence, which is not complementary to the template, for example, a sequence indicating a position of a restriction enzyme and a promoter sequence (McPherson and Moller, 2000). The oligonucleotide primer may have a nucleotide sequence completely complementarily to a specific portion of the template, i.e., a nucleotide sequence that does not have a mismatched base sequence.

The term “priming” or “annealing” indicates that an oligonucleotide or primer is complementarily positioned on the template nucleic acid. Based on the priming or annealing, a polymerase polymerizes nucleotides from the end of the primer to produce a nucleic acid molecule having a sequence complementary to the template or a portion of the template. Thus, the term “priming sequence” used herein refers to a sequence substantially complementary to the targeted nucleic acid and having a length sufficient to provide specificity for producing only the targeted nucleic acid after the elongation from the primer.

The length of the oligonucleotide primer should be sufficient for initiating the polymerization of the elongated product in the presence of the polymerase and may be selected by one of ordinary skill in the art based on temperature, template nucleic acid sequence, application fields, sources of the primer, and the like. In particular, the length of the primer may be determined in consideration of the temperature for annealing and the number of nucleotides sufficient for providing specificity for hybridization with the template. As the number of nucleotides constituting the oligonucleotide primer increases, specificity for the targeted sequence increases and the annealing temperature for the template increases. The priming sequence may have at least 6 nucleotides that are the minimum requirement for the primer annealing. The primer may have 12 to 100 nucleotides, for example, 15 to 40 nucleotides, but the length of the primer is not limited thereto.

The “photolabile/photocleavable protecting group” refers to a chemical moiety that is attached to a reactive functional group to protect the functional group from undesired reaction until it is removed by light irradiation or exposure to light. The protected functional group may be, for example, a hydroxy group. In this case, the photolabile protecting group may be photocleavable hydroxy group-protecting group. The photolabile protecting group is attached to a 3′-hydroxy group of a 3′ terminal nucleoside of the primer and prevents the 3′-hydroxy group from being polymerized with another nucleotide monomer until light is irradiated.

In addition, the term “photolabile/photocleavable compound” is understood as a compound to which the photolabile protecting group is attached, for example, a nucleoside or nucleotide monomer, an oligonucleotide, or the like. The term “photolabile compound” used herein refers to a nucleoside or nucleotide monomer or an oligonucleotide in which a photolabile protecting group is attached to or binds to a hydroxy group of position 3 of ribose or deoxyribose thereof. In particular, the photolabile/photocleavable primer refers to an oligonucleotide primer having a 3′-OH group of the 3′ terminal protected by a photolabile protecting group. Deprotection refers to a chemical reaction wherein the photolabile protecting group is removed or cleaved so that a reactive functional group is exposed and converted into a chemically reactive state. According to the current embodiment, the deprotection indicates a chemical reaction wherein the photolabile protecting group is removed from the primer so that the 3′-hydroxy group is restored and the primer is activated for the polymerization. The deprotection may be performed under constant conditions which do not damage or change molecules having the functional group.

The photolabile protecting group used herein may be a moiety that is physically stable at a temperature range where denaturation occurs during a polymerization of a nucleic acid, for example, during a PCR. In addition, the protecting group may be removed by the light irradiation having an adjusted wavelength for a relatively short period of time, for example, for several seconds to several minutes to accurately control the polymerization. In addition, the photolabile protecting group that is removed from the primer may not produce reactive metabolites or by-products capable of causing background in analysis data.

Any photolabile protecting group that satisfies the above conditions may be used in the preparation of the photolabile oligonucleotide primer. According to the current embodiment, the photolabile protecting group may include a nitro aromatic compound such as a 2-nitrobenzyl derivative (G. Ciamician and P. Silber, Chem. Ber. 1901, 34, 2040) or an o-nitrobenzyloxy derivative, a benzoin derivative (M. C. Pirrung and L. Fallon, J. Org. Chem. 1998, 63, 241), or benzyl sulfonyl, but is not limited thereto. The nitro aromatic compound may include 6-nitroveratryloxycarbonyl (NVOC), 2-nitrobenzyloxycarbonyl (NBOC), 2-(3,4-methylenedioxy-2-nitrophenyl)oxycarbonyl (MeNPOC), 2-(2-nitrophenyl)propyloxycarbonyl (NPPOC), 2-(2-nitrophenyl)ethylsulfonyl (NPES), 2-(2-nitrophenyl)propylsulfonyl (NPPS), 2-(3,4-methylenedioxy-2-nitrophenyl)propyloxycarbonyl (MeNPPOC), 2-(5-phenyl-2-nitrophenyl)-propyloxycarbonyl (PhNPPOC), o-nitrobenzylthioethyloxycarbonyl (NBTEOC), o-nitrophenylaminocarbonyl (NPAC), o-nitrophenoxycarbonyl (NPOC), α-methyl-8-nitronaphthylmethoxycarbonyl (MeNMOC), o-nitrophenylthioethyloxycarbonyl, or α,α-dimethyldimethoxybenzyloxycarbonyl (DDZ). According to another embodiment of the present invention, the photolabile protecting group may be 1-pyrenyl methyloxycarbonyl (PYMOC), anthracenyl-methyloxycarbonyl (ANMOC), dimethoxytritriyl (DMT), and the like, in addition to nitro aromatic compound.

The photolabile protecting group may be introduced into a 3′-OH group of a nucleotide by using a method disclosed by M. Beier, et al., Helv Chim Acta 2001, 84, 2089. The article identified above is incorporated into the specification by reference.

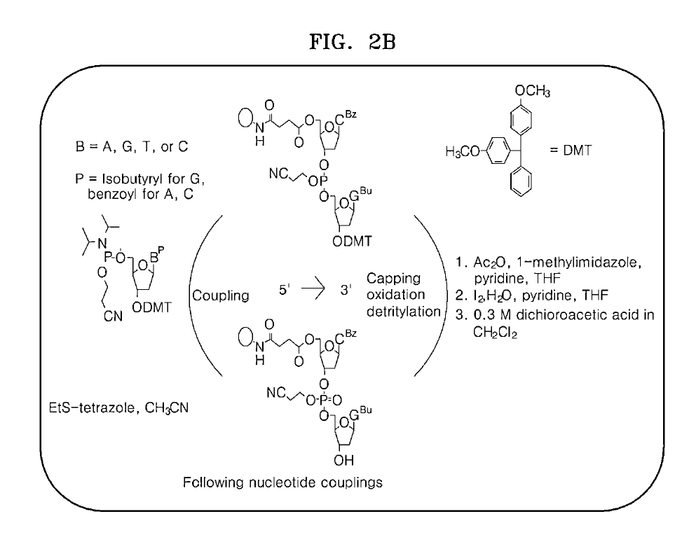

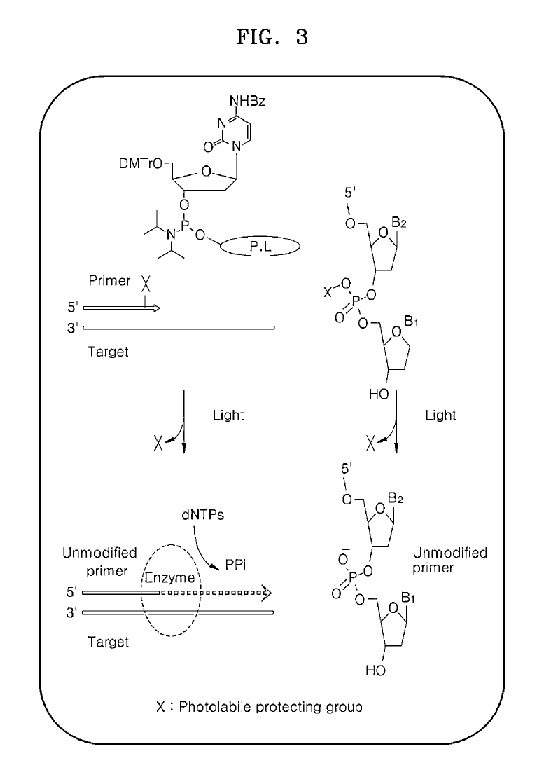

For example, the photolabile oligonucleotide primer used herein may be prepared by condensation-polymerizing a nucleoside monomer having a 3′-OH group protected by the photolabile protecting group with a new oligonucleotide including a priming sequence by using a method commonly used to synthesize oligonucleotides. The polymerization may be performed by a phosphoramidite reaction that is known in the art. In this case, the nucleoside monomer protected by the photolabile protecting group is a 5′-phosphoramidite nucleoside monomer. A dialkylamine group of the 5′-phosphoramidite monomer is substituted with a 3′-terminal hydroxy group of the new oligonucleotide so that the condensation-polymerization is performed in a 5′->3′ direction (FIG. 2).

The synthesis of the photolabile oligonucleotide primer may be performed in a solution or on a solid substrate. If the synthesis is performed in a solution, the concentration of the new oligonucleotide may be in the range of 0.001 M to 1.0 M and the concentration of the nucleoside monomer having a protected 3′-OH group may be in the range of about 1.1 to about 2 equivalents based on the oligonucleotide. If the synthesis is performed on the solid substrate, a linker that links the solid substrate with the new oligonucleotide may be used. When the synthesis is terminated, the product may be isolated from the solid substrate by using a standard method, for example, by treating the resultant with, for example, an ammonium hydroxide concentrate for 0.5 to 16 hours. In addition, the produced oligonucleotide primer may be purified by using at least one of known methods such as ion exchange chromatography, reverse phase chromatography, and precipitation in a solvent.

A primer or primer pair comprising a 3′-hydroxyl group protected by a photolabile protecting group, as described for use in the method of amplifying a nucleic acid, also is provided as an independent aspect of the invention. According to an embodiment of the present invention, the primer and 3′-terminal nucleoside of the photolabile oligonucleotide primer may be represented by Formula 1 below:

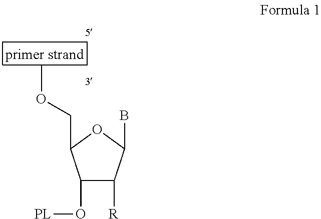

In Formula 1, B may be adenine, cytosine, guanine, thymine, uracil, or modified nucleic acid base,

R may be a hydrogen atom, a halogen atom, a hydroxy group, —OR₁, or —SR₁, wherein R₁ may be a C₁-C₆ alkyl group, a C₂-C₆ alkenyl group, an acetal group, or a silyl ether, and

PL may be a photolabile protecting group as defined above with respect to the method of amplifying a nucleic acid; and “primer strand” is an oligonucleotide.

In Formula 1, B, which is a pyrimidine or purine base constituting, may include natural nucleic acid bases such as adenine, guanine, cytosine, thymine, and uracil, and variants thereof. According to an embodiment of the present invention, B may be selected from the group consisting of adenine, cytosine, guanine, and thymine if the targeted sequence is a deoxyribonucleotide chain, or may be selected from the group consisting of adenine, cytosine, guanine, and uracil if the targeted sequence is a ribonucleotide chain. According to another embodiment of the present invention, B may be variants of adenine, cytosine, guanine, thymine, or uracil which may form complementary base pairs with the targeted nucleic acid sequence. Examples of the variants may include 7-deazaguanidine, 7-deaza-8-azaguanidine, 5-propynylcytosine, 5-propynyluracil, 7-deazaadenine, 7-deaza-8-azaadenine, 7-deaza7-oxopurine, 6-oxopurine, 3-deazaadenosine, 2-oxo-5-methylpyrimidine, 4-oxo-5-methylpyrimidine, 2-aminopurine, 5-fluorouracil, 2,6-diaminopurine, 8-aminopurine, 4-triazolo-5-methylthymine, 4-triazolo-5-methyluracil, hypoxanthine, 5-methylcytosine, and 5-amino-4-imidazolecarboxylic acid amide, but are not limited thereto. Any other known variants of nucleic acid bases may also be used.

The nucleic acid bases may be isolated or protected by a suitable protecting group. In particular, the pyrimidine or purine base having a primary amino group, e.g., adenine, cytosine, and guanine, may be protected by a protecting group having a carbonyl group. The carbonyl protecting group may include phenoxyacetyl, dimethyl formamidino radical, but is not limited thereto. A base specific-protecting group may also be used. For example, a benzoyl radical or a p-nitrophenyl ethoxy carbonyl (p-NPEOC) radical may be used for adenine, a p-NPEOC radical, an isobutyroyl protecting group, or a p-nitrophenylethyl (p-NPE) protecting group may be used for guanine, and a p-NPEOC radical, a benzoyl protecting group, or an isoburyroyl protecting group may be used for cytosine.

R may be selected from the group consisting of a hydrogen atom, a halogen atom, a hydroxy group, and a protected hydroxyl or thiol group (—OR₁ or —SR₁). If R is —OR₁ or —SR₁, R₁ may be a hydroxyl or thiol protecting group that is commonly used in a nucleotide compound. According to an embodiment of the present invention, R₁ may be a protecting group selected from the group consisting of an alkyl group, an alkenyl group, an acetal group, or a silylether group, for example, a C₁-C₆ alkyl group, a C₂-C₆ alkenyl group, an acetal group, or a silylether group. The term “alkyl” used herein generally refers to a C₁-C₂₀ linear or branched monovalent saturated hydrocarbon radical, for example, a C₁-C₈ alkyl group. The term “alkenyl” used herein refers to an unsaturated hydrocarbon radical including at least one carbon-carbon double bond which is a linear, branched, chain, or cyclic radical, for example, a C₂-C₈ alkenyl group. For example, R may be any one radical selected from the group consisting of an O-methyl radical, an O-ethyl radical, an O-allyl radical, an O-tetrahydropyranyl radical, an O-methoxytetrahydropyranyl radical, and an O-t-butyldimethylsilyl radical.

In the method of amplifying nucleic acids, according to the current embodiment, the “reactants for nucleic acid amplification” refers to a composition including essential ingredients for the amplification of nucleic acids, i.e., a primer, a nucleic acid polymerase, a template nucleic acid, and reaction substrates such as nucleotide triphosphates (NTPs). The types and amounts of the primer, the polymerase, and the NTPs may be adjusted by one of ordinary skill in the art according to the types and lengths of the template nucleic acid to be amplified, amplification techniques, and purposes of the amplification. The reactants for nucleic acid amplification used in the method of amplifying nucleic acids, according to the current embodiment, may have the same composition as reactants used for common nucleic acid amplification, except that the photolabile oligonucleotide primer is used.

The method of amplifying nucleic acids, according to the current embodiment includes exposing the reactants for nucleic acid amplification to electromagnetic radiation in a wavelength range capable of removing the photolabile protecting group.

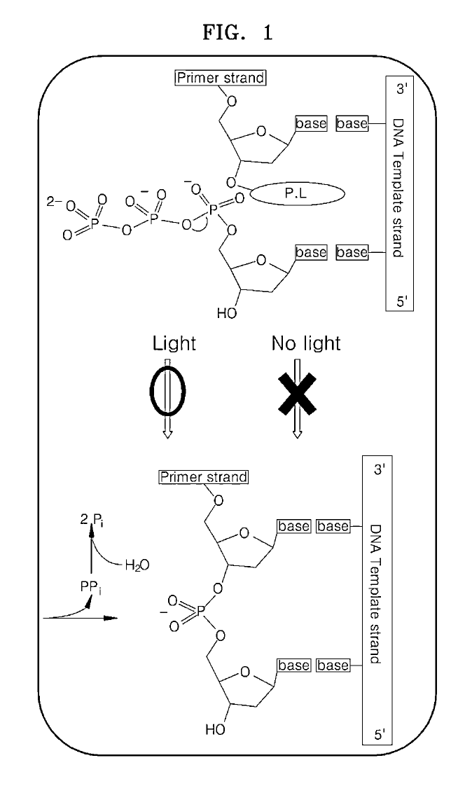

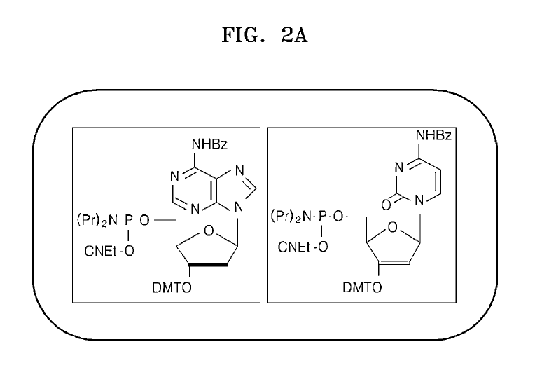

The exposure to the electromagnetic radiation initiates polymerization by deprotecting the 3′-terminal of the oligonucleotide primer. Once exposed to the electromagnetic radiation having wavelengths absorbed by the photolabile protecting group, the protecting group of the primer is removed by photolysis so that the 3′-OH group, which is an active functional group is restored, and thus the nucleotide synthesis may be initiated (FIG. 1). According to general techniques for nucleic acid amplification, it is difficult to accurately control polymerization since the polymerization is controlled based on physical properties, chemical reactivity, and enzyme activity by controlling temperature. According to the current embodiment, the amplification of nucleic acids may be accurately controlled, regardless of the temperature or the activation of the polymerase, by irradiating light to the reactants for nucleic acid amplification including the photolabile primer at a desired point of time.

The technique of controlling polymerization by activating the primer by light irradiation may be widely used for various fields that require optimized polymerization conditions. For example, the method of amplifying nucleic acids, according to the current embodiment, may be used to reduce non-specific amplification products. According to an embodiment of the present invention, non-specific amplification of nucleic acids caused by mispriming may be considerably reduced by irradiating amplification reactants comprising the photolabile primer with electromagnetic radiation under high annealing stringency conditions, for example, at a high temperature. This method may be efficiently applied to multiplex PCRs in which the reduction of non-specific amplification is required, or to the amplification of nucleic acids with a long sequence or high GC content.

According to another embodiment of the present invention, the method of amplifying nucleic acids may be performed by using PCR that includes exposing PCR reactants comprising the photolabile primer to electromagnetic radiation when the temperature of a reaction system reaches a predetermined level in order to inhibit non-specific amplification. For example, the exposure to the electromagnetic radiation may be performed after the reaction system reaches a thermal denaturation temperature of the template. Since a DNA polymerase is generally active at room temperature, amplification of nucleic acid may occur whenever essential ingredients for the polymerization are combined, even before heating the reaction system. Accordingly, in the mixing step before heating, non-specific annealing may occur due to the formation of a primer-dimer or a secondary structure of a template nucleic acid, e.g., hairpin loop, and thus by-products of the amplification may be produced. According to the current embodiment, however, since the primer remains inactive and the amplification does not occur until being exposed to light by-products caused by non-specific annealing and amplification may be reduced by irradiating light after a DNA double helix is denatured by the thermal denaturation. The present inventors have found that specificity of the amplification is significantly increased, compared with PCR performed using a general primer to which the photolabile protecting group is not attached, by preparing PCR reactants using the photolabile primer and irradiating the reactants with light at elevated temperature before initiating the PCR thermal cycle (Example 3).

Such efficient control of amplification by light irradiation is performed by simple chemical modification of the primer. Thus, the photolabile protecting group that binds to the primer does not influence stability of the reactants. In addition, since enzymes, antibodies, and other chemical additives (oil used as a shield), wax, or various organic solvents as promoters such as PEG, DMSO, and glycerol) are not required for inhibiting non-specific amplification, reactants and devices for amplifying nucleic acids, and processes of amplifying nucleic acids may be simply prepared. In addition, the absence of additives leads to increased reproducibility, reduced occurrence of background (non-DNA-containing band) and decreased production of impurities, and thereby improving detection and analysis efficiencies.

According to the current embodiment, the wavelengths of the electromagnetic radiation may vary according to one of ordinary skill in the art based on the absorption wavelength of the photolabile protecting group. In consideration of the stability of the nucleic acid molecules, the wavelength range of the electromagnetic radiation may be about 300 nm to about 450 nm, for example, within ultraviolet/visible (UV/VIS) light regions. According to an embodiment of the present invention, the wavelength of the electromagnetic radiation used for the deprotection of the photolabile primer may be in the range of about 330 nm to about 400 nm. For example, if the photolabile protecting group is 2-(3,4-methylenedioxy-2-nitrophenyl)oxycarbonyl (MeNPOC), 2-(2-nitrophenyl)propyloxycarbonyl (NPPOC), 2-(2-nitrophenyl)ethylsulfonyl (NPES), 2-(2-nitrophenyl)propylsulfonyl (NPPS), 2-(3,4-methylenedioxy-2-nitrophenyl)propyloxycarbonyl (MeNPPOC), 2-(5-phenyll-2-nitrophenyl)-propyloxycarbonyl (PhNPPOC), dimethoxybenzoinyl oxycarbonyl (DMBOC), or dimethyltrityl (DMT), the primer may be deprotected by irradiating electromagnetic radiation in a wavelength range of about 340 nm to about 380 nm.

The irradiation of the electromagnetic radiation or exposure thereto may be performed by using a light source that emits light having wavelengths capable of removing the photolabile protecting group. Examples of the light source include a high pressure mercury lamp, an ultra-high pressure mercury lamp, a metal halide lamp, a halogen lamp, a gallium lamp, a xenon lamp, an incandescent source, a laser beam, and a laser diode, but are not limited thereto. According to an embodiment of the present invention, the exposure to the electromagnetic waves may be performed by using a high pressure mercury lamp that emits I-line with a wavelength of about 365 nm.

If a collimated light source is not used, a multi-layer mask or a thick mask may be used to prevent scattering of light. If required, electromagnetic radiation that is not within a desired wavelength range may be blocked. For example, light with a short wavelength less than 300 nm, which may damage base moiety of the nucleic acid, may be blocked using a PYREX® filter.

The irradiation of light may be performed in the presence of a hydroxylic solvent or a protic solvent such as an aqueous solvent, an alcohol solvent, an aqueous-alcohol solvent, or an aqueous-organic solvent, as a photolabile medium. The photolabile medium may selectively include a nucleophilic scavenger such as hydrogen peroxide. In general, the photolysis may be performed in neutral or basic pH conditions. As such, the method of removing the protecting group by irradiating the photolabile primer or the reaction mixture for nucleic acid amplification with electromagnetic radiation may be performed according to a known photolysis deprotection method.

According to the current embodiment, the irradiation of the electromagnetic radiation may be performed at a desired point of time. For example, the irradiation of the electromagnetic waves may be performed whenever the template nucleic acid, the primer, the polymerase, and the reaction substrate (NTP), all of which are essential ingredients for the nucleic acid amplification, are mixed together. The electromagnetic radiation may be uniformly or non-uniformly irradiated once or several times.

The time period for the irradiation may be selected by one of ordinary skill in the art in consideration of a half-life and absorption wavelengths of the photolabile protecting group, the light source, the solvent, and concentrations of the reactants. According to the method of amplifying nucleic acids of the current embodiment, the protecting group may have a half-life in the range of several seconds to several minutes, and the irradiation of the electromagnetic waves may be conducted for about 10 seconds to about 10 minutes. According to an embodiment of the present invention, the amplification of nucleic acids may be performed by using polymerase chain reaction (PCR). In order to increase amplification efficiency, a photolabile protecting group having a half-life of about 1 minute or less, for example, MeNPOC, PYMOC, and ANMOC, may be used. In this case, the amplification speed may be increased when compared to a hot-start PCR that requires a pre-heating period for about 15 minutes to about 40 minutes, and by-products resulted from the retardation of amplification may be prevented.

The method of amplifying nucleic acids, according to the current embodiment, may be applied to a variety of nucleic acid amplifications known in the art. The amplifications may include PCR, ligase chain reaction, polymerase/ligase chain reaction, Gap-LCR (WO90/01069), high fidelity PCR (using proofreading enzymes), 3SR (Kwoh, et al., PNAS, U.S.A., 86:1173 (1989)) and NASBA (U.S. Pat. No. 5,130,238), but are not limited thereto. For example, the nucleic acid amplification may be performed by using PCR. Examples of the PCR may include allele-specific PCR, assembly PCR, asymmetric PCR, colony PCR, emulsion PCR, fast PCR, gap extension ligation PCR (GEXL-PCR), helicase-dependent amplification, hot-start PCR, intersequence-specific (ISSR) PCR, inverse PCR, ligation-mediated PCR, linear-after-the-exponential PCR (LATE-PCR), methylation-specific PCR (MSP), multiplex ligation-dependent probe amplification (MLPA), multiplex PCR, nested PCR, overlap-extension PCR, PAN-AC, quantitative PCR (Q-PCR), quantitative real-time PCR (QRT-PCR), real-time PCR, rapid amplification of cDNA ends (RACE PCR), single molecule amplification PCR (SMA PCR), thermal assymetric interlaced PCR (TAIL-PCR), touch down PCR, single molecule amplification PCR (SMA PCR), and reverse transcription PCR (RT-PCR), but are not limited thereto.

The method of amplifying nucleic acids, according to an embodiment of the present invention, is performed by PCR, the method including: providing PCR reactants that include an oligonucleotide primer (pair) having a 3′-hydroxy group protected by a photolabile protecting group; exposing the PCR reactants to electromagnetic radiation with a wavelength capable of removing the photolabile protecting group from the primer; denaturing a template DNA; annealing the primer to the template DNA; and elongating the annealed primer.

The term “PCR reactants” indicates reactants for nucleic acid amplification essential for the polymerization by the PCR, which may include a template nucleic acid to be amplified, a DNA polymerase, primer (pair), Mg²⁺, and nucleoside triphosphates (NTPs), such as deoxynucleoside triphosphates (dNTPs). The reactants may be prepared by simultaneously mixing all ingredients or by mixing some of the ingredients and then subsequently adding the other ingredients. Used herein, the PCR reactants have the same composition as that of commonly used PCR reactants, except that the oligonucleotide primer (pair) having the 3′-OH group protected by the photolabile protecting group is used. The types and contents of the essential ingredients and additives may be adjusted by one of ordinary skill in the art according to the purpose of the amplification.

The exposure to the electromagnetic radiation includes deprotecting the 3′-terminal by irradiating electromagnetic waves having wavelengths suitable for cleaving the photolabile protecting group from the photolabile primer. The wavelength of the electromagnetic radiation may be adjusted by one of ordinary skill in the art according to the absorption wavelength of the photolabile protecting group. The wavelength may be selected from a UV/VIS region or a range of about 300 nm to about 450 nm, for example, about 340 nm to about 380 nm, in consideration of the stability of bases of the nucleic acid. The exposure time may be adjusted by one of ordinary skill in that art based on the types of the photolabile protecting group, the light source, the solvent, and concentrations of the substrates. During the PCR, a time period of the electromagnetic irradiation may be reduced using a protecting group having a relatively short photolysis half-life in order to increase the amplification speed.

The exposure of the electromagnetic radiation may be performed at a temperature range of about 30° C. to about 100° C. The temperature may vary according to the light exposure time, the length of the template, the intended level of specificity, or the like. According to an embodiment of the present invention, the electromagnetic radiation may be irradiated at the elevated temperature of about 30° C. to about 60° C. According to another embodiment of the present invention, the electromagnetic radiation may be irradiated at a high temperature in the range of about 90° C. to about 97° C. after an initial thermal denaturation for separating the DNA double-stranded structure.

The denaturation may be performed by using heat or ultrasound, or the like, for isolating the double helix of the template DNA. According to an embodiment of the present invention, the DNA maybe thermally denatured by heating the PCR reactants to a desired temperature. The denaturation temperature may be adjusted by one of ordinary skill in the art according to the length of DNA and the content of the guanine/cytosine. As the temperature increase, the double helix is isolated to single-strands. However, the activity of the polymerase may be reduced at a temperature higher than a predetermined level. Thus, the denaturation temperature may be in the range of about 90° C. to about 97° C. if a Taq DNA polymerase is used. Before the PCR thermal cycle including the thermal denaturation-annealing-elongation is initiated, an initial thermal denaturation may be performed for a desired period of time, for example, for several seconds to 10 minutes. The thermal denaturation repeated during the thermal cycle may be performed for a shorter period of time, for example, about 1 second to about 30 seconds.

According to an embodiment of the present invention, the electromagnetic radiation may be irradiated simultaneously with the initial thermal denaturation or separately. When performed separately from the initial thermal denaturation, the irradiation of the electromagnetic radiation may be performed before heating or after the temperature reaches the denaturation temperature. However, if the polymerization is prevented at a low temperature to reduce the production of the non-specific amplification products, the irradiation may be performed after the initial thermal denaturation, or at the same time with the denaturation. The mispriming with targeted sequence, the formation of secondary structure of the template DNA, and the formation of primer dimer are considerably prevented under high temperature conditions which enable the thermal denaturation and thus non-specific amplification is reduced.

The hybridization or annealing may be performed under conditions where the oligonucleotide primer is hybridized with a portion of the template to form double-strands. The annealing conditions such as temperature and time may vary according to one of ordinary skill in the art in consideration of the base sequence of the primer (GC content), a melting point (Tₘ) of the primer, and a length of the primer. According to an embodiment of the present invention, the annealing may be performed at a temperature in the range of about 50° C. to about 72° C., for example, about 55° C. to about 62° C. As the annealing temperature increases, the specific hybridization indicating completely matching with the targeted sequence increases. Thus, high stringency conditions (high temperature) under which the mismatch between the targeted nucleic acid sequence and the primer is reduced may be selected to reduce the non-specific amplification.

In the elongation of the primer, temperature and time may be adjusted by one of ordinary skill in the art according to temperature in which the DNA polymerase is activated, the concentration of the template DNA, the size of the amplified fragments. For example, if a thermostable Taq DNA polymerase is used, the elongation may be performed at a temperature in which the activity of the enzyme is optimized, i.e., at about 68° C. to about 75° C., for example, at about 72° C., for about 30 seconds to about 1 minute. If the PCR product has a small size or the concentrations of the reactants are low, the elongation time may be increased. In addition, during the final cycle, the elongation may be performed for a sufficient time, for example, about 2 minutes to about 10 minutes.

According to an embodiment of the present invention, the PCR includes: preparing PCR reactants that include a template DNA, a photolabile oligonucleotide primer pair, a Taq DNA polymerase, a magnesium salt, and dNTP; exposing the PCR reactants to electromagnetic radiation at a temperature of about 30° C. to about 100° C.; initially denaturing DNA at a temperature of about 90° C. to about 97° C.; repeating thermal cycles consisting of thermal denaturation-annealing-primer elongation; and performing additional elongation at a temperature of about 68° C. to about 75° C.

The PCR cycle including the thermal denaturation-annealing-primer elongation may be repeated 10 times to 50 times, but the number of repetition is not limited thereto. If required, at least two of the above thermal cycle operations may be performed simultaneously, being combined as one step. According to an embodiment of the present invention, the annealing and the primer elongation may be simultaneously performed at the same temperature, for example, at about 60° C. In this embodiment, the PCR thermal cycle includes only two operations, i.e., a thermal denaturation and an annealing/elongation.

According to another embodiment of the present invention, at least two types of targeted sequences may be amplified using at least two types of primer pairs in the same reaction. A plurality of targeted sequences may be amplified using a known multiplex PCR (Chamberlain, et al., 1988). In general, it is very difficult to set PCR conditions for amplifying more than 10 types of targeted sequences. According to the method of amplifying nucleic acids, according to an embodiment of the present invention, the polymerization is initiated by irradiating light when the temperature is sufficiently high for annealing where complete DNA-DNA matches are possible, so that amplification specificity is improved. Thus, the method is suitable for optimizing multiplex PCR conditions. The method of amplifying nucleic acids applied to the multiplex PCR are the same as defined above, except that more than two types of targeted nucleic acid sequences and more than two different primer pairs are used.

According to another embodiment of the present invention, the method of amplifying nucleic acids may be a method of selectively amplifying the targeted nucleic acid sequence using mRNA as a template. The method may include reverse transcription of mRNA and amplification of a nucleic acid using cDNA prepared by the reverse transcription. The reverse transcription may include contacting an oligonucleotide dT primer that is hybridized with a poly A tail of the template mRNA with the mRNA under conditions suitable for synthesis of template-derived enzymatic deoxyribonucleic acid and producing a complementary DNA strand by reverse-transcription of the mRNA with which the oligonucleotide dT primer is hybridized.

According to an embodiment of the present invention, provided is a composition for amplifying a nucleic acid including an oligonucleotide primer or primer pair having a 3′-hydroxy group protected by a photolabile protecting group.

The composition for amplifying nucleic acids according to the current embodiment includes a composition that is commonly used for the amplification, except that the photolabile oligonucleotide primer (pair) is added to the composition. Thus, the composition for amplifying nucleic acids may include a nucleic acid polymerase, reaction substrates constituting a targeted nucleic acid sequence (nucleoside triphosphates (NTPs) or dNTPs), and a buffer solution including a magnesium ion (Mg²⁺) source in addition to the photolabile oligonucleotide primer or primer pair, For example, the composition for amplifying nucleic acids may be a composition for PCR. The composition for PCR has a similar composition to PCR reactants commonly used in the art, except that the photolabile oligonucleotide primer is used. According to an embodiment of the present invention, the composition for PCR may include a photolabile oligonucleotide primer (pair) hybridized with a portion of the targeted sequence, a DNA polymerase having thermal resistance at a temperature where the amplification is performed, a magnesium salt, and a dNTP.

The amount of the photolabile primer in the composition for PCR may be adjusted by one of ordinary skill in the art according to concentration of a sample. For example, about 1 to about 1000 pmol of the primer may be used in a 50 μl of reactants.

The DNA polymerase may include thermostable DNA polymerases obtained from various bacteria. Examples of the thermostable DNA polymerase include polymerases obtained from Thermus aquaticus (Taq), Thermus ruber, Thermus thermophilus (Tth), Thermus filiformis, Thermis flavus, Thermus lacteus, Thermus rubens, Thermococcus literalis, Bacilus stearothermophilus, Methanothermus fervidus or Pyrococcus furiosus (Pfu), but are not limited thereto. For example, the thermostable DNA polymerase may be a Taq polymerase. Most of the polymerases may be isolated from bacteria or commercially available.

The composition for PCR may be prepared by using a buffer solution including a magnesium ion source and dNTP.

According to an embodiment of the present invention, the magnesium ion source contained in the composition indicates a magnesium salt capable of providing Mg²⁺ when it is melted or isolated under the conditions for the amplification. The magnesium salt may include magnesium chloride, magnesium acetate, magnesium sulfate, and the like, but is not limited thereto. The content of the magnesium salt may be in the range of about 1 to about 20 mM, for example, 2 to 10 mM, in the PCR reactants.

The composition may include deoxynucleotside triphosphates (dNTP) (dATP, dGTP, dCTP, and dTTP) as substrates for DNA elongation. In addition, analogues of dNTP may be used as a substrate for the DNA polymerase. Examples thereof include 7-deaza-dGTP and dUTP including an amino group, but are not limited thereto. Excessive dNTP may be contained in the PCR reactants. The ‘excessive’ refers to an amount by which the amplification is not substantially limited. According to an embodiment of the present invention, the final concentration of the dNTP may be in the range of about 0.1 mM to about 3.0 mM, for example, about 0.2 mM to about 2.7 mM in the PCR reactants. If the final concentration of dNTP is about 4 mM or greater, the activity of the polymerase may be reduced by about 20 to 30%.

The buffer used in the composition for PCR according to the current embodiment may include Tricine, Bicine, MOPS, HEPES, TAPS, TES, PIPES, MES, or Tris-chloride, but is not limited thereto. In general, a buffer solution including Tricine or phosphate (for example, sodium phosphate and potassium phosphate) may be used. If the reaction is performed at a high temperature, a Bicine buffer solution, the pH of which does not change with temperature, may be used. The final concentration of the buffer in the PCR reactants may be in the range of about 5 mM to about 100 mM, for example, about 20 mM to about 50 mM, but is not limited thereto. In addition, the final pH of the PCR reactants may be in the range of about 6.0 to about 9.5, for example, about 7.0 to about 9.2, but is not limited thereto. According to an embodiment of the present invention, a buffer solution including 25 to 50 mM of potassium phosphate having a pH of 7.0 to 8.0 may be used.

According to another embodiment of the present invention, a dried product (i.e., a composition in dried form) for PCR including photolabile oligonucleotide primer or primer pair is provided. The dried product for PCR may be prepared by drying a composition for PCR including the photolabile oligonucleotide primer (pair) as defined above in a reaction tube. The dried product may further include, for example, one or more of a DNA polymerase, a magnesium ion source, and 4 types of dNTPs, if required, a template nucleic acid, a probe, a fluorescent dye, and PPase, in addition to the photolabile oligonucleotide primer. According to another embodiment of the present invention, a kit for PCR including photolabile oligonucleotide primer or primer pair is provided. The kit for PCR may be a kit including a mixture of photolabile oligonucleotide primer or primer pair as defined above, a DNA polymerase, a magnesium salt, and 4 types of dNTPs, or a kit in which the ingredients are mixed after the initiation of the PCR.

Other compounds or compositions typically used in PCR reactions also may be included in the dried product or kit, although, in one embodiment, the dried product or kit consists essentially of or consists of any one or more of the foregoing.

According to another embodiment of the present invention, provided is a device for amplifying nucleic acids including a light-transmitting sample receiving unit and a light-irradiating unit for performing nucleic acid amplification using a photolabile oligonucleotide primer.

The device for amplifying nucleic acids may be a PCR device. The PCR device has a similar constitution to a device commonly used for PCR, except that the light-transmitting sample receiving unit and the light-irradiating unit that irradiates electromagnetic waves to a sample are disposed. The light-transmitting sample receiving unit may be a sample receiving container formed of a transparent material, for example, a tube or a pipe. The light-irradiating unit may be an optical system that is commonly used to irradiate a sample contained in the light-transmitting sample receiving unit with electromagnetic radiation. For example, the light-irradiating unit may include: a light source that emits electromagnetic radiation; a lens that is disposed next the light source and collimates the light; and a filter unit that is disposed next the lens and transmitting light having a desired wavelength.

The light source may be a light source emitting electromagnetic radiation with a wavelength that may be absorbed by the photolabile protecting group, for example, with a wavelength range of 300 nm to 450 nm. The light source may be selected from the group consisting of a laser, a LED, a metal halide lamp, a halogen lamp, an incandescent lamp, a gallium lamp, a high pressure mercury lamp, and an ultra-high pressure mercury lamp, but is not limited thereto. The light emitted by the light source passes the collimating lens and the filter unit which are disposed sequentially. The filter unit passes light with a wavelength range absorbed by the photolabile protecting group and blocks light having wavelengths that are not absorbed by the photolabile protecting group. Accordingly, the filter unit may include two blocking filters that block light having wavelengths longer than the upper limit or shorter than the lower limit of a pass-band.

The PCR device may further include an irradiation control unit disposed between the light-transmitting sample receiving unit and the light-irradiating unit so that light is irradiated when the temperature of the device reaches the initial thermal denaturation temperature after the PCR reaction. The irradiation control unit may include a sensor that senses temperature of the inside of the light-transmitting sample receiving unit when the temperature reaches a predetermined thermal denaturation temperature and a signal transmitting system that sends a signal to the light source of the light-irradiating unit to irradiate electromagnetic waves. The irradiation control unit may send a signal to the light source when the temperature of the inside of the light-transmitting sample receiving unit reaches, for example, 94° C.

According to another embodiment of the present invention, there is provided phosphoramidite nucleoside analogues represented by Formula 2 below which may be used for the preparation of the photolabile primer, the method of amplifying nucleic acids, and the composition or the kit for amplifying nucleic acids using the photolabile

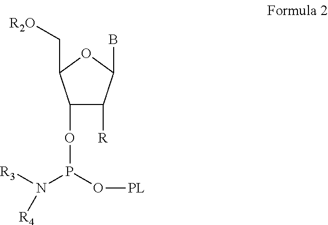

In Formula 2, B may be adenine, cytosine, guanine, thymine, uracil, or modified nucleic acid base,

R may be a hydrogen atom, a halogen atom, a hydroxy group, —OR₁, or —SR₁, wherein R₁ may be a C₁-C₆ alkyl group, a C₂-C₆ alkenyl group, an acetal group, or a silyl ether,

R₂ is H or a hydroxy protecting group,

R₃ and R₄ are each independently an aliphatic C₁-C₈ alkyl group, a C₂-C₈ alkenyl group, an aryl group, or an aralkyl group; or R₃, R₄ and the nitrogen bound thereto form an azaheterocyclyl group, and

PL is a photolabile protecting group.

In the phosphoramidite nucleoside of Formula 2, the B, R, R₁, and PL are as defined above.

In R₂, the “hydroxy protecting group” is widely known in the art. Examples of commonly used hydroxy protecting group are disclosed by Green, et al., Protective Groups in Organic Synthesis (1991), John Wiley & Sons, Inc., 309-405 pp, the disclosure of which is incorporated herein in its entirety by reference. The hydroxy protecting group used herein may include a C₁-C₆ alkyl group, a C₁-C₆ alkoxy alkyl group, an acyl group, an aralkyl group, an alkylsulfonyl group, an arylsulfonyl group, a silyl group substituted with a C₁-C₆ alkyl group, an alkoxy carbonyl group, an aryloxycarbonyl group, an aralkoxy carbonyl group, or a tetrahydropyranyl group, but is not limited thereto. For example, the hydroxy protecting group may be an acetyl group, a benzoyl group, a dimethoxybenzoyl group, a trimethylsilyl group, a t-butyldimethylsilyl group, a benzyloxycarbonyl group, a dimethoxytrityl group or a tetrahydropyranyl group.

The aliphatic alkyl group and alkenyl group of R₃ and R₄ are as defined above. If each of R₃ and R₄ are an alkyl group, they may be isopropyl groups. The term “aryl” used herein indicates an aromatic hydrocarbon ring (for example, phenyl), an aromatic hydrocarbon ring system fused to at least one aromatic hydrocarbon ring (for example, naphthyl or anthracenyl), or an aromatic hydrocarbon ring system fused to at least one non-aromatic hydrocarbon ring (for example, 1,2,3,4-tetrahydronaphthyl). The term “aralkyl” used herein refers to an aryl group bound to a nitrogen atom by an alkyl group, for example, a C₁-C₆ alkyl group, (for example, benzyl). The term “heterocyclyl” used herein includes a heteroaryl group and a heteroallylcyclyl group. The heteroaryl group refers to a 5-membered or 6-membered aromatic ring including at least one heteroatom selected from the group consisting of S, N, and O. The heteroallylcyclyl group refers to a 5-membered or 6-membered non-aromatic ring including at least one heteroatom selected from the group consisting of S, N, and O. Examples of the heterocyclyl group include morpholinyl, piperidinyl, piperazinyl, thiomorpholinyl, pyrrolidinyl, thiazolidinyl, tetrahydrothienyl, azetidinyl, tetrahydrofuryl, dioxanyl, thienyl, pyridyl, thiadiazolyl, oxadiazolyl, indazolyl, furan, pyrolyl, imidazolyl, benzimidazolyl, indolyl, tetrahydroindolyl, azaindolyl, indazolyl, quinolinyl, imidazopyridinyl, purine, pyrolol[2,3-d]pyrimidinyl, and pyrazolo[3,4-d]pyrimidinyl, but are not limited thereto.

The alkyl, alkenyl, aryl, or heterocyclyl group may be substituted with at least one substituent. Examples of the substituent include a halogen atom, a hydroxy group, a cyano group, a nitro group, a C₁-C₆ alkyl group, a halogenated C₁-C₆ alkyl group (for example, trifluoromethyl), a C₁-C₆ alkoxy group, a halogenated C₁-C₆ alkoxy group, and a benzyl group, but are not limited thereto.

The compound of Formula 2 may be used in the preparation of oligonucleotides using a known method of synthesizing phosphoramidite. In particular, the compound is a photolabile phosphoramidite nucleoside in which a photolabile protecting group is attached to oxygen of a phosphate moiety in 3′-phosphoramidite and may be used in the preparation of the photolabile primer for amplifying nucleic acids. As such, if a primer having a phosphate group of a 3′-terminal nucleoside to which a photolabile protecting group is attached is hybridized or annealed with a nucleic acid having a complementary base sequence, the photolabile protecting group is positioned outside the double helix structure to form spatial barriers so that a polymerase could not approach the phosphate group. If electromagnetic radiation having adjusted wavelengths are irradiated to a sample including the primer, the photolabile protecting group is cleaved, so that the polymerase approaches the phosphate group of the 3′-terminal to initiate polymerization (FIG. 2). Accordingly, the compound of Formula 2 may contribute to precisely control polymerization by light irradiation and reduce products of non-specific amplification.

According to another embodiment of the present invention, provided is a photolabile oligonucleotide primer having a 3′-terminal into which the compound of Formula 2 is introduced. According to another embodiment of the present invention, provided is a method of amplifying nucleic acids including exposing reactants for amplification including the primer to electromagnetic radiation capable of removing the photolabile protecting group. According to another embodiment of the present invention, provided are a composition and kit for amplifying nucleic acids including the primer (pair). The primer, the method of amplifying nucleic acids, the composition, and the kit may be the same as those described above, except that the photolabile nucleoside introduced into the 3′-terminal of the primer has the structure of Formula 2, and may be modified by one of ordinary skill in the art.

Unless otherwise defined, all technical terms used herein have the same meaning as commonly understood by one of ordinary skill in the art to which this invention belongs. Example embodiments are to cover all modifications, equivalents, and alternatives falling within the scope of the invention. All the cited references are incorporated herein by reference in their entireties.

The present invention will be described in further detail with reference to the following preparation examples and experimental examples.

### Example 1

### Synthesis of Oligonucleotide Primer Having 3′-Hydroxy group Protected by Photolabile Protecting Group

Oligonucleotides including a primer set of a forward primer having a 3′-terminal to which a photolabile nucleoside phosphoramidite monomer is introduced and having a sequence of 5′-ACGAGTAGATGCTCAATA-3′ (mecA_Forward, SEQ ID NO: 1) and a reverse primer having a sequence of 5′-GGAATAATGACGCTATGAT-3′ (mecA_Reverse, SEQ ID NO: 2) and a probe FAM having a sequence of 5′-CCAATCTAACTTCCACATACCATCT-BHQ1-3′ (mecA_probe SEQ ID NO: 3), and a primer set of a forward primer having a sequence of 5′-TGATGCGGGTTGTGTTAATTGA-3′ (MREJ Forward, SEQ ID NO: 4) and a reverse primer having a sequence of 5′-TCCACATCTCATTAAATTTTTAAATTATACACA-3′ (MREJ Reverse, SEQ ID NO: 5), and a probe FAM having a sequence of 5′-AGAGCATTTAAGATTATGCG-BHQ1-3′ (MREH probe SEQ ID NO: 6) were synthesized. The synthesis of the oligonucleotide was performed according to a standard phosphoramidite chemistry protocol by Genotech, Co., Ltd. Briefly, the oligonucleotide was prepared by condensation-polymerizing a nucleoside monomer having a 3′-OH group protected by the photolabile protecting group with a new oligonucleotide including a priming sequence. The polymerization was performed by phosphoramidite reaction, wherein the nucleoside monomer protected by the photolabile protecting group was a 5′-phosphoramidite nucleoside monomer. A dialkylamine group of the 5′-phosphoramidite monomer was substituted with a 3′-terminal hydroxy group of the new oligonucleotide so that the condensation-polymerization was performed in a 5′->3′ direction. This process is illustrated in FIG. 2.

### Example 2

### Synthesis of Phosphoramidite Nucleoside Monomer Having 3′-Phosphate Group Protected by Photolabile Protecting Group

A phosphoramidite nucleoside was prepared by reaction between a nucleoside monomer having a protected 5′-OH group and a phosphorochloridite to which a photolabile protecting group is attached in the presence of an organic base. 3′-Photocaged DNA phosphoramidite synthesis was used, as follows: (1) Synthesis of 6-nitroveratryloxybis(diisopropylamino)phosphine: Bis(diisopropylamino)chlorophosphine (1 mmol, 1 eq), and diisopropylethylamine (1 mmol, 1 eq) were added to methylene chloride. 6-Nitroveratryl alcohol (1 mmol, 1 eq) was added dropwise to the solution under nitrogen and the mixture was stirred for 2 hr at 0° C. Then, the reaction mixture was extracted with brine and methylene chloride and dried with MgSO4, following filtration and evaporation. The residue was purified by silica gel chromatography. (2) Synthesis of 5′-O-(4,4′-dimethoxy)-N6-Pac-adenosine-3′-6-nitroveratryloxybis(diisopropylamino)phosphine: 5′-O-(4,4′-dimethoxy)-N6-Pac-adenosine (1 mmol, 1 eq), and 5-ethylthio-1H-tetrazole (1 mmol, 1 eq) were dissolved in dry acetonitrile, and 6-nitroveratryloxybis(diisopropylamino)phosphine (1 mmol, 1 eq) was added dropwise for min under nitrogen. After the mixture was stirred for 3 hr at 0° C., the reaction mixture was quenched with water and evaporated. Then, the residue was extracted with brine and EtOAc and dried with MgSO₄, following filtration and evaporation. The desired product was obtained by silica gel chromatography. All chemicals were commercially purchased from Sigma-Aldrich, ChemGenes. All solvents used were distilled, and the silica gel for column chromatography was supplied as 300-400 meshes.

The synthesized phosphoramidite nucleoside monomer was polymerized with a new oligonucleotide according to a standard phosphoramidite chemistry protocol to prepare a photolabile oligonucleotide primer having a protected 3′-phosphate group.

### Example 3

### Inhibition of Non-Specific Amplification in PCR

3-1. Preparation of Composition for PCR Including Photolabile Oligonucleotide Primer.

A PCR reaction solution having the following composition was prepared. TaKaRa Z-Taq™ (2.5 units/μl), 10×Z-Taq Buffer (including 30 mM of Mg²⁺), and dNTP Mixture (2.5 mM of each of dATP, dGTP, dCTP, and dTTP) were used. 1 μM of a forward primer, 1 μM of a reverse primer, and 400 nM of a probe were added thereto. The amount of the Taq DNA polymerase used in the PCR was adjusted according to a manual. The reaction solution was filtered using a 0.2 μm filter, and different concentrations of the template DNA were added thereto. A template used in the PCR was genomic DNA (gDNA) extracted from MRSA cells. The extraction of the gDNA was performed using a G-spin Genomic DNA extraction kit (cell/tissue) manufactured by Intron Biotechnology Co. A gene to be amplified by PCR had a size of 100 to 200 bp.

A photolabile primer set having a protected 3′-OH group prepared according to Example 1 was used in test groups, a general primer set having the same priming sequence was used in a negative control group, and a known thermolabile primer set having the same priming sequence was used in a positive control group in a PCR reaction solution.

3-2. Measurement of Inhibition of Non-Specific Amplification by Photolabile Primer

PCR was performed by adding 1.2 μl of each of the reaction solutions to a TMC 2000 (thermocycler) (Samsung Electronics Co., Ltd.). Basic reaction conditions are as follows:

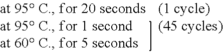

The test group and control groups were exposed to light at different temperatures before an initial thermal denaturation at 95° C. A mercury lamp irradiating I-line (λ=365 nm) was used as a light source. If light was not irradiated, the reaction solution was prepared and PCR was performed in a dark condition. After the amplification was terminated, amplification products were cooled at low temperature. The PCR products were analyzed in a 1% agarose gel by electrophoresis.

As a result of electrophoresis, the polymerization of nucleic acids was not performed in the dark condition. On the other hand, PCR products having sizes in the range of about 100 to about 200 bp were produced in the groups exposed to light. In particular, it was identified that non-specific amplification products was considerably reduced in the test groups where the primer set having the 3′-terminal protected by the photolabile protecting group was used, compared with the negative and positive control groups. In addition, it was observed that as the exposure temperature increases, non-specific amplification further decreased.

As described above, according to the one or more of the above embodiments of the present invention, the polymerization may be precisely controlled by deprotecting the active site by irradiation of light if the nucleic acid amplification is performed using the photolabile compound. In particular, in the method of amplifying nucleic acids using the photolabile primer, mispriming and non-specific amplification caused by the mispriming may be prevented by irradiating light under conditions for achieving high stringency annealing.

Furthermore, according to the method of amplifying nucleic acids using the photolabile compound, the polymerization of the nucleic acid may be initiated at a desired point of time regardless of the thermal denaturation of the nucleic acids. Thus, in addition to inhibiting non-specific amplification as an alternative method to a hot start PCR, the method may have various applications in controlling nucleic acid polymerization.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An oligonucleotide primer or primer pair having a 3′-hydroxy group protected by a photolabile protecting group, wherein the photolabile group can be removed by electromagnetic radiation.

2. The primer or primer pair of claim 1, wherein the primer or primer pair comprises the following formula: 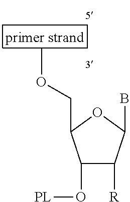
wherein B is adenine, cytosine, guanine, thymine, uracil, or modified nucleic acid base,
R is a hydrogen atom, a halogen atom, a hydroxy group, —OR₁, or —SR₁, wherein R₁ may be a C₁-C₆ alkyl group, a C₂-C₆ alkenyl group, an acetal group, or a silyl ether, and
PL is a photolabile protecting group that can be removed by electromagnetic radiation,
and the primer strand is an oligonucleotide.

3. The primer or primer pair of claim 1, wherein the electromagnetic radiation has a wavelength of about 300 nm to about 450 nm.

4. The primer or primer pair of claim 1, wherein the photolabile protecting group is a 2-nitrobenzyl derivative, an o-nitrobenzyloxy derivative, a benzoin derivative, or a benzyl sulfonyl derivative.

5. The primer or primer pair of claim 1, wherein the photolabile protecting group is 6-nitroveratryloxycarbonyl (NVOC), 2-nitrobenzyloxycarbonyl (NBOC), 2-(3,4-methylenedioxy-2-nitrophenyl)oxycarbonyl (MeNPOC), 2-(2-nitrophenyl)propyloxycarbonyl (NPPOC), 2-(2-nitrophenyl)ethylsulfonyl (NPES), 2-(2-nitrophenyl)propylsulfonyl (NPPS), 2-(3,4-methylenedioxy-2-nitrophenyl)propyloxycarbonyl (MeNPPOC), 2-(5-phenyl-2-nitrophenyl)-propyloxycarbonyl (PhNPPOC), o-nitrobenzylthioethyloxycarbonyl (NBTEOC), o-nitrophenylaminocarbonyl (NPAC), o-nitrophenoxycarbonyl (NPOC), α-methyl-8-nitronaphthylmethoxycarbonyl (MeNMOC), o-nitrophenylthioethyloxycarbonyl, α,α-dimethyldimethoxybenzyloxycarbonyl (DDZ), 1-pyrenyl methyloxycarbonyl (PYMOC), anthracenyl-methyloxycarbonyl (ANMOC), or dimethoxytritriyl (DMT).

6. A composition comprising the primer or primer pair of claim 1.

7. The composition of claim 6, further comprising a DNA polymerase, a Mg²⁺ source, and deoxynucleoside triphosphates (dNTPs).

8. The composition of claim 6, wherein the composition is in a dried form.

9. A kit for PCR comprising the primer or primer pair of claim 1.

10. A method of amplifying a nucleic acid, the method comprising exposing PCR reactants comprising a template nucleic acid and a primer or primer pair according to claim 1 to electromagnetic radiation in a wavelength range capable of removing the photolabile protecting group from the primer, and amplifying the template nucleic acid.

11. The method of claim 10, wherein the nucleic acid is amplified by PCR, and the method comprises:
exposing the PCR reactants to electromagnetic radiation of ultraviolet/visible (US/VIS) light to remove the photolabile protecting group from the primer;
denaturing a template DNA;
annealing the primer to the template DNA; and
elongating the annealed primer.

12. The method of claim 11, wherein the photolabile protecting group is a 2-nitrobenzyl derivative, an o-nitrobenzyloxy derivative, a benzoin derivative, or a benzyl sulfonyl derivative.

13. The method of claim 11, wherein the photolabile protecting group of the primer is 2-(3,4-methylenedioxy-2-nitrophenyl)oxycarbonyl (MeNPOC), 2-(2-nitrophenyl)propyloxycarbonyl (NPPOC), 2-(2-nitrophenyl)ethylsulfonyl (NPES), 2-(2-nitrophenyl)propylsulfonyl (NPPS), 2-(3,4-methylenedioxy-2-nitrophenyl)propyloxycarbonyl (MeNPPOC), 2-(5-phenyl I-2-nitrophenyl)-propyloxycarbonyl (PhNPPOC), dimethoxybenzoinyl oxycarbonyl (DMBOC), or dimethyltrityl (DMT).

14. The method of claim 11, wherein the PCR reactants are exposed to the electromagnetic radiation at a temperature of about 30° C. to about 100° C.

15. The method of claim 14, wherein the PCR reactants are exposed to the electromagnetic radiation at a temperature of about 90° C. to about 97° C.

16. The method of claim 11, wherein the electromagnetic radiation comprises I-line waves.

17. The method of claim 11, wherein the electromagnetic radiation has a wavelength of about 300 nm to about 450 nm.

18. A device for amplifying a nucleic acid using the primer or primer pair of claim 1, the device comprising
a light-transmitting sample receiving unit,
and a light-irradiating unit, wherein the light-irradiating unit comprises
a light source that emits electromagnetic radiation in ultraviolet/visible (UV/VIS) light regions,
a lens that is disposed next to the light source and collimates the light,
and a filter unit that is disposed next to the lens and that transmits light of a desired wavelength;
and wherein the light-transmitting sample receiving unit comprises a sample receiving container formed of a transparent material through which electromagnetic radiation in UV/VIS light regions passes.

19. The device of claim 18, further comprising an irradiation control unit disposed between the light-transmitting sample receiving unit and the light-irradiating unit, wherein the irradiation control unit comprises a sensor that senses the temperature of the inside of the light-transmitting sample receiving unit and a signal transmitting system that sends a signal to the light source according to the sensed temperature.

20. A phosphoramidite nucleoside represented by the following formula: 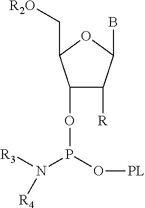
wherein B is adenine, cytosine, guanine, thymine, uracil, or a modified nucleic acid base,
R is a hydrogen atom, a halogen atom, a hydroxy group, —OR₁, or —SR₁, wherein R₁ is a C₁-C₆ alkyl group, a C₂-C₆ alkenyl group, an acetal group, or a silyl ether,
R₂ is H, a hydroxy protecting group, or a nucleotide chain;
R₃ and R₄ are each independently an aliphatic C₁-C₈ alkyl group, a C₂-C₈ alkenyl group, an aryl group, or an aralkyl group; or R₃, R₄ and the nitrogen bound thereto form an heterocyclyl group, and
PL is a photolabile protecting group that can be removed by electromagnetic radiation.
